(19) United States
(12) Patent Application Publication
(10) Pub. No.: US 2003/0113715 A1
(43) Pub. Date: Jun. 19, 2003

(54) Method for cloning polyketide synthase genes

(76) Inventor: Daniel Santi, San Francisco, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/765,541

(22) Filed: Jan. 19, 2001

Related U.S. Application Data
(60) Provisional application No. 60/177,285, filed on Jan. 21, 2000.

(51) Int. Cl. C12Q 1/68; C07H 21/04; C12P 19/34; C12N 15/74

(52) U.S. Cl. 435/6; 435/91.2; 435/455; 536/24.3; 435/474

(57) ABSTRACT

A method for obtaining “perfect probes” for type I modular polyketide synthase (PKS) or non-ribosomal peptide synthase (NRPS) gene clusters enables the identification of all such gene clusters in a genome. By sequencing small fragments of a random genomic DNA library containing one or more modular PKS or NRPS gene clusters, and identifying which fragments emanate from PKS or NRPS genes and knowing the approximate sizes of the genome and the target gene cluster, one can predict the frequency that a PKS or NRPS gene fragment will be present in the library sequenced.

### CROSS-REFERENCE

The present application claims priority to U.S. patent application Serial No. 60/177, 285, filed Jan. 12, 2000, incorporated herein by reference.

### FIELD OF THE INVENTION

The present invention relates to the fields of biology, molecular biology, chemistry, medicinal chemistry, agriculture, and animal and human health science.

### BACKGROUND OF THE INVENTION

Polyketide synthases (PKS) catalyze the biosynthesis of a class of microbial natural products known as polyketides (for a recent review, see Cane, CHREAY, 1997, pp. 2463-2705), many of which are important pharmaceutical agents. Of the three major types of PKSs, the modular type I PKSs consist of multiple large polyfunctional proteins, and catalyze the biosynthesis of most of the non-aromatic polyketides. In 1990-91, DNA sequencing of the genes encoding the erythromycin PKS revealed the remarkable finding that the genes and the encoded proteins have a modular architecture (Cortés et al., Nature 348 (1990) 176-178; Donadio et al., Science 252 (1991) 675-679).

The prototypical modular PKS, exemplified by the erythromycin PKS (FIG. 1), is encoded by a cluster of contiguous genes, and has a loading module of ˜2 to 4 kb, a linear organization of ˜6 modules (although the number may be in some cases as high as 20) of ˜4 to 5.5 kb each, and a small thioesterase (TE) or releasing domain. Each module contains three to six domains that are homologous to other PKS domains of like function. All modules possess ketosynthase (KS), acyltransferase (AT) and acylcarrier protein (ACP) domains that are necessary for the two-carbon (ketide) unit elongation of the polyketide chain.

In addition, modules may contain one to three enzyme domains that modify the oxidation state of the ketide unit: a keto reductase (KR) domain, KR and dehydratase (DH) domains, or a KR, DH, and enoyl reductase (ER) domains. The composition of domains within a module serves as a “code”for the structure of each two-carbon unit of the polyketide. The order of the modules in a PKS specifies the sequence of the two-carbon units. The number of modules determines the number of two-carbon units or size of the polyketide chain.

Non-ribosomal peptide synthase (NRPS) enzymes also have a modular architecture. Each NRPS module contains an adenylation (A), condensation (C) and thiolation (T) domain that together specify the amino acid added to the growing oligopeptide. Accessory domains may include domains for epimerization, N-methylation, or oxidation (Marahiel et al., Chemical Reviews 97 (1997) 2651-2673; Marahiel, Chem. Biol. 4 (1997) 561-567). As with the PKSs, the order, specificity and number of NRPS modules determine the amino acid sequence and size of the oligopeptide.

The identification and isolation of a PKS or NRPS gene cluster is a prerequisite to heterologous expression of the polyketide or non-ribosomal peptide (or compounds that have elements of each, such as epothilone) and to genetic engineering of the PKS or NRPS to produce novel “unnatural”natural products. The approach usually involves identification of clones within a genomic cosmid library that contain the desired PKS or NRPS gene by hybridization with DNA probes from other PKS or NRPS gene clusters or by gene fragments amplified by PCR of genomic DNA using degenerate primers. Because the amino acid sequence of individual domains of modular PKSs or NRPSs is usually quite similar, such approaches are often successful. However, because probes or primers are often imperfect, PKS or NRPS gene clusters may be missed.

Moreover, organisms often contain multiple PKS and/or NRPS gene clusters, so that probes or primers may reveal some PKS or NRPS gene clusters, but not uncover the one sought. This can result in ill-fated efforts devoted towards an incorrect gene cluster, as reported in pursuit of PKS gene clusters from Streptomyces hygroscopicus (Ruan et al., Gene (1997) 1-9), S. cinnamonensis (Arrowsrnith et al., Mol Gen Genet 234 (1992) 254-264), and others (Hopwood, Chemical Reviews 97 (1997) 2465-2497).

The cloning and characterization of PKS and NRPS genes would be considerably easier if there were a method to generate a set of DNA fragments that contain representatives from each and every modular PKS and/or NRPS gene cluster in a genome. The probes could serve as a tool for identifying, cloning, or otherwise manipulating PKS and/or NRPS gene clusters in a genome and provide a means for estimating the fraction of the genome containing PKS and/or NRPS sequences. The present invention provides such a method.

### SUMMARY OF THE INVENTION

The present invention provides a method to assemble a set of DNA fragments that contain representatives (“perfect probes”) from each and every modular PKS and/or NRPS gene cluster in a genome. The probes can be used to identify PKS and/or NRPS gene clusters in a genome and to estimate the fraction of the genome containing PKS and/or NRPS sequences. The method involves sequencing small fragments of a uniform size random genornic DNA library and identifying fragments of PKS or NRPS gene clusters by homology to known PKS or NRPS genes. Knowing the approximate genome and PKS or NRPS gene cluster sizes, one can predict the frequency with which an identifiable PKS or NRPS gene fragment will be present in the library sequenced (Lander et al., Genornics 2 (1988) 231-239). A computer-simulation of the approach is applied to the known single PKS and NRPS gene clusters in the Bacillus subtilus genome (Kunst et al., Nature 390 (1997) 249-256). For illustrative purposes, the method is applied to identify PKS gene cluster fragments in a strain of Sorangium cellulosum that produces epothilone. While the specific examples provided are directed to modular PKS gene clusters, the approach is also directly applicable to NRPS gene clusters.

Thus, the invention provides a method for generating a perfect probe for any PKS or NRPS gene in an organism, the method comprising the steps of:

(a) generating a genomic library of vectors containing insert DNA from said organism;

(b) generating nucleotide sequence information from said vectors;

(c) comparing said nucleotide sequence information generated with sequence information from a known PKS or NRPS gene;

(d) identifying vectors with insert DNA that contains nucleotide sequences from a PKS or NRPS gene,

wherein said insert DNA that contains nucleotide sequences from a PKS or NRPS gene is a perfect probe for said PKS or NRPS gene.

The perfect probes thus generated can then be used to identify clones in a genomic library, such as a BAC or cosmid library containing very large inserts of genomic DNA of the organism, that contain the PKS or NRPS genes of interest. With these perfect probes, one can identify a particular PKS or NRPS gene of interest or all of the PKS or NRPS genes in the organism. The perfect probes are also useful as primers for amplification.

In a preferred embodiment, sequence information is obtained from at least the number of clones in the library that, based on the average insert size of the clones, the size of the genomic DNA of the organism of interest, and the average size of a PKS and/or NRPS gene cluster, ensures that at least one clone will contain an insert derived from at least one PKS or NRPS gene cluster. This number of clones is called a “micro-library.” In a more preferred embodiment, the number of clones sequenced is larger than the number of clones in the micro-library. For example, by sequencing two, three, four, and five times the number of clones in the micro-library, one can increase the probability of identifying all PKS or NRPS gene clusters in the organism. In a preferred embodiment, at least the number of clones required to achieve an 80, to 95, to 99% probability of identifying all PKS or NRPS gene clusters in the organism is sequenced.

The sequence information obtained using this method is useful in constructing oligonucleotide probes and primers complementary to at least a portion of a PKS or NRPS gene cluster of interest. In one embodiment, probes are constructed and then used to identify DNA fragments, recombinant vectors, or host cells comprising all or a portion of the desired PKS or NRPS gene cluster. In another embodiment, primers are constructed that are used to amplify a DNA or RNA derived from the PKS or NRPS gene cluster of interest. In another embodiment, the probe or primer is employed to clone the PKS or NRPS gene cluster of interest and determine the nucleotide sequence of one or more genes in the gene cluster.

These and other embodiments, modes, and aspects of the invention are described in more detail in the following description, the examples, and claims set forth below.

### BRIEF DESCRIPTION OF THE DRAWING

FIG. 1 shows the modular organization of a prototypical PKS, the 6-dEB PKS. Functional domains of the modules of each of the three polypeptides from the DEBS PKS gene cluster are shown, along with intermediate polyketide chains produced. Stepwise synthesis of 6-dEB begins at DEBS1 and ends with cyclization by the TE domain to yield 6-dEB which is further functionalized (not shown) to yield erythromycin.

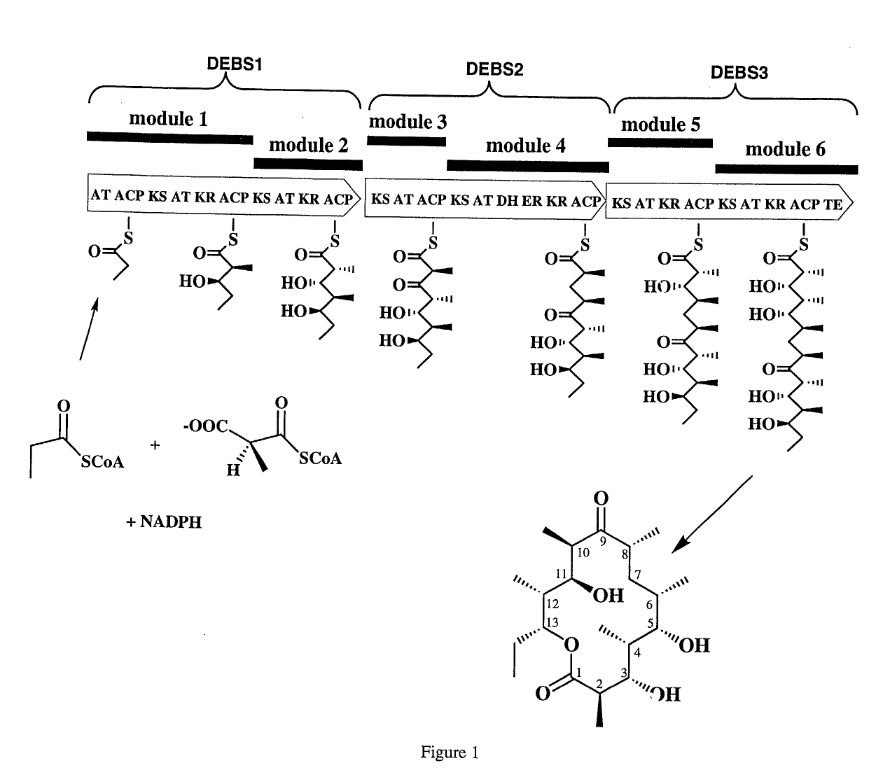

### DETAILED DESCRIPTION OF THE INVENTION

If a microbial genome is cloned as a library of small, uniform size random fragments, the frequency of PKS sequences in the library reflects that in the genome. As defined here, a “micro-library” consists of the random number of clones that on the average contains one fragment from a single PKS gene. Because PKSs are highly homologous, sequencing 300 to 500 bases provides sufficient amino acid sequence to identify a fragment as part of a PKS gene. By sequencing a statistically sufficient number of clones and identifying those that contain a PKS gene fragment, the fraction of the genome that contains PKS gene sequences can be estimated. Further, by assuming a size for the target PKS gene cluster, sufficient coverage of the micro-library will insure the presence of a representative fragment from any PKS gene cluster in the genome. From Poisson distribution, three- and four-fold sequence coverage of a micro-library would provide 95 and 98% probabilities, respectively, that a fragment from a PKS gene cluster would be obtained (Table 1) (Lander, 1988).

TABLE 1
Probability of identifying a PKS fragment by random
sequencing of a genomic micro-library

| | Probability of |
|---|---|
| Coverage of micro- | identifying a PKS |
| library | gene fragment^{a} |
| 0.50 | 39% |
| 1.0 | 63% |
| 2.0 | 87% |
| 3.0 | 95% |
| 4.0 | 98% |
| 5.0 | 99% |

^{a}Determined by Poisson distribution (Lander, 1988) and assumes that every PKS gene fragment present will be identified as such.

Thus, if a prototypical modular PKS gene cluster were ˜40 kb, it would represent ˜0.4% of a 10 Mb genome of its source microorganism, and one fragment of the PKS gene would be found in a micro-library of 250 clones (i.e. 0.4%) containing random 1,000 bp genomic fragments. If n 40 kb PKS gene clusters were present in the genome, then on average, n PKS fragments would be found in every 250 clones. With knowledge of the genome size and assumptions of the average size of a PKS gene cluster, the identified PKS gene fragments could be used to estimate the total number of PKS genes in the genome. For example, if DNA sequencing revealed three PKS fragments per 250 fragments, the PKS genes would occupy ˜1.2% of a 107 bp genome, corresponding to about three prototypical 40 kb PKS gene clusters in the genome.

Most important, modular PKS fragments identified as above would serve as a collection of “perfect probes”to assist in the identification of PKS gene clusters from a library of large fragment clones in cosmid or BAC vectors.

Within limits, the size of the fragments in the micro-library should have little effect on the approach, so long as they are identifiable PKS fragments and are sufficiently uniform in size to allow the statistical calculations needed. For example, because type I modular PKS genes are large and contiguous, clones containing DNA fragments in the range of 1 to 5 kb should be readily identifiable as containing a PKS gene fragment by end sequencing. Indeed, larger fragments would require a smaller component library to be sequenced and thus may in practice be advantageous; larger fragments would also offer tools needed for directed gene disruption studies.

A computer-simulation of the approach directed towards the known PKS gene cluster of B. subtilis was performed. The B. subtilis genome consists of 4,214 kb, and contains a single 38.9 kb PKS gene cluster [Accession U11039, M97902]. The PKS genes therefore represent 0.92% of the genome and if the B. subtilis genome were cloned as 4,214 fragments of 1 kb, 39 (or 1 in 108 examined) would contain a PKS fragment. The B. subtilis genome was fragmented in silico to give a library of 1 kb fragments. A random number generator was used to sample the set of 1 kb fragments, and the first 500 bp of each were probed against the genome sequence to determine whether it contained a PKS gene sequence. After processing 400 fragments (˜4-fold coverage of the 108-fragment microlibrary) 4.4 PKS gene fragments were found. This suggests that 1.1% of the B. subthlus genome is PKS sequence, which is in good agreement with the actual value of 0.92%.

The NRPS genes represent another modular system in which individual translated fragments are readily recognizable by homology to known NRPSs. A computer-simulation was also performed to identify the 39.8 kb NRPS gene cluster (Z34883) that generates plipastatin in B. subtilis (Steller et al., Chem. Biol. 6 (1999) 31-41; Tsuge et al., Antimicrob Agents Chemother 43 (1999) 2183-2192). Here, the plipastatin genes represent 0.94% of the genome, and in a genonuc library of 1 kb fragments, 1 in 106 would posses a NRPS fragment. As before, a library of 1 kb fragments of the B. subtilus genome was randomly sampled, and the first 500 bp of each probed against the genome sequence to determine whether they contained fragments of the plipastatin gene. After 400 fragments (˜4-fold coverage of the 106-fragment microlibrary), were processed, 4.2 NRPS gene fragments were found. This suggests that 1.05% of the B. subtilus genome is NRPS sequence, in good agreement with the actual value of 0.94%.

In another illustrative embodiment of the invention, experiments were undertaken to isolate the PKS gene cluster that encodes the epothilone PKS in Sorangium cellulosum SMP44. Epothilone is a new anti-cancer agent, and cloning of the PKS could be used to produce epothilone in a more advantageous host and to prepare novel analogs by engineering the PKS genes. At the outset of these studies, no PKS genes had been isolated from this strain of S. cellulosum.

Initially, degenerate PCR primers designed from conserved KS sequences of several PKSs and fatty acid synthases were used, and two fragments from genomic DNA were isolated. The isolated fragments were used as probes for a genomic cosmid library and provided two positives; a third positive cosmid was isolated from overlap with one of the initial two. Mapping and sequencing of these clones revealed a PKS gene cluster with >70 kb DNA that was designated the tmbA gene cluster; however, the module arrangement was inconsistent with that predicted from the structure of epothilone (see U.S. patent application Ser. No. 09/144, 085, filed 31 Aug. 1998, and U.S. Pat. No. 6, 090, 601). In a second attempt (see PCT publication No. 00/031247), a different set of degenerate PCR primers designed from KS sequences of soraphen (Schupp et al., J. Bacteriol 177 (1995) 3673-3679; Ligon et al., U.S. Pat. No. 5, 716, 849) and erythromycin (Donadio et al., Science 252 (1991) 675-679) PKSs were used to isolate nine unique PKS gene fragments of S. cellulosum DNA. Three were from the aforementioned tmbA PKS gene cluster, two were subsequently shown to be derived from the epothilone gene cluster, and four were unknown. These experiments indicated that there were at least 3 PKS gene clusters in the organism.

When it became apparent that the S. cellulosum SMP44 genome contained several PKS gene clusters, there was concern that additional effort might be wasted pursuing an incorrect PKS gene cluster, prompting the creation of a new approach.

An estimation of the approximate size of a type I modular PKS gene cluster can be made from the structure of the polyketide coupled with the assumption that each ketide (two-carbon) unit of the polyketide backbone is derived from the activities of a module of ˜5 kb of DNA. The 16-membered macrolactone of epothilone has a starting unit and 8 ketide units that are predicted to be synthesized by a 9-module PKS, corresponding to about 45 kb of coding DNA; the actual size of PKS genes in the epothilone PKS gene cluster has recently been determined to be ˜50 kb. The related Myxococcus xanthus genome is ˜10⁷ bp, and the epothilone PKS gene cluster was estimated to represent about 0.45% of the genome of S. cellulosum. From this, a micro-library of -220 kb clones of a 1 kb fragment micro-library should contain ˜1 epothilone PKS gene fragment. A random library of small fragments from S. cellulosum genomic DNA was produced, and readable sequences for 495 randomly chosen clones was obtained (˜2.2-fold coverage of the micro-library), and the translated amino sequences probed against the NCBI non-redundant database. Sixteen fragments had translated sequences homologous to domains of known PKSs; as shown in Table 2, there were four ACPs, four ATs, six KSs, one ER, and one KS-AT boundary.

TABLE 2
Perfect polyketide probes for S. cellulosum SMP44
obtained from sequencing 495 clones of genomic DNA

| | Clone | Gene Cluster | domain |
|---|---|---|---|
| 1 | ala08mx | epo PKS | ACP |
| 2 | ala10mx | epo PKS | AT |
| 3 | alb02mx | tmba PKS | ACP |
| 4 | ald11mx | Unknown PKS | KS |
| 5 | ale05mx | Unknown PKS | KS |
| 6 | a2a04mx | Unknown PKS | ER |
| 7 | a2a10mx | Unknown NRPS | A |
| 8 | a3b06mx | Unknown NRPS | A |
| 9 | a3b11mx | Unknown NRPS | A |
| 10 | a3e06mx | Unknown PKS | KS |
| 11 | a3f04mx | Unknown PKS | AT |
| 12 | a4a08mx | Unknown PKS | KS |
| 13 | a4a11mx | Unknown PKS | KS |
| 14 | a4h01mx | Unknown PKS | AT |
| 15 | a5c02mx | Unknown PKS | AT |
| 16 | a5e08mx | Unknown PKS | KS |
| 17 | a6c05mx | Unknown PKS | AT/KS |
| 18 | a7b10mx | Unknown PKS | ACP |
| 19 | a7c03mx | Unknown NRPS | C |
| 20 | a7d01mx | Unknown PKS | ACP |

One of these sixteen sequence fragments corresponded to the aforementioned tmbA PKS gene cluster, two to the epothilone PKS gene cluster and the remaining 13 originated from thus far unidentified PKS gene clusters. The identification of epothilone fragments in 1 per ˜246 fragments sequenced is in good agreement with the predicted 1 per 222. In addition to the PKS gene fragments, four NRPS sequences were identified in the library that corresponded to three adenylation domains and 1 condensation domain.

The data obtained in this study allow estimates of the PKS gene content of Sorangium cellulosum SMP44 genome. The finding of 16 PKS fragments in 495 sequences (3.2%) suggests that PKS gene clusters represent ˜3.2% of the S. cellulosum SMP44 genome, or a total of ˜320 kb. Assuming an average size of 40 to 50 kb for a modular PKS gene cluster, one can predict there could be six to eight PKS gene clusters in this organism. Alternatively, from the genes thus far sequenced, the tmbA and epothilone genes correspond to a total of about 120 kb, leaving ˜200 kb of unidentified PKS gene sequences in this organism.

The present method involves sequencing of a small, uniform sized fragment library of genomic DNA, and identification of fragments of type I modular PKS (or NRPS) genes. With the genome size known, the frequency of PKS fragments in the library allows an approximation of the fraction of the genome that corresponds to type I modular PKS genes; with further assumptions of the size of a typical gene cluster, the approximate number of PKS gene clusters can be estimated. Moreover, the method provides “perfect probes” that can be used to identify and isolate every modular PKS gene cluster in a genome. In one application of the method, a mixture of perfect probes is hybridized with colonies of a large fragment cosmid DNA library to reveal all colonies that contain PKS gene clusters. Alternatively, individual probes can be used to identify individual unique modular PKS gene clusters.

If an organism has multiple PKS gene clusters, there is a possibility that significant time and effort will be expended pursuing the incorrect cluster. For example, as indicated above, the probability of selecting the epothilone gene cluster by chance among all present in the Sorangium cellulosum SMP44 genome was only about 1 in 6 to 8. A complete collection of perfect probes, as described here, can serve as tools to assist in the identification of a target PKS gene cluster prior to the investment of major efforts. For instance, in an organism with multiple PKS gene clusters, MRNA transcripts coordinately produced with a secondary metabolite (Proctor et al., Fungal Genet Biol 27 (1999) 100-112) could be identified by probing with individual PKS “perfect-probes”. The positive probes could then be used to identify the corresponding complete PKS gene clusters in a large fragment library. Minimally, this would eliminate cryptic PKS gene clusters from consideration that might otherwise occupy experimental effort. Additionally, if the fragment library is of sufficient size (≧2 kb), fragment DNAs of PKS genes could be directly used in gene disruption experiments to identify PKS genes necessary for secondary metabolite production. The focus of the specific experimental study described herein was directed towards the epothilone modular PKS gene cluster, and the method may not be as practical for the isolation of smaller, non-modular PKS gene clusters, which could require sequencing of a very large micro-library of DNA fragments.

Although the approach described here requires the sequencing of hundreds of fragments of genomic DNA, the investment is small when compared to sequencing and assembling an entire PKS gene cluster with the risk that it is not the one sought. Further, with the capillary DNA sequencers available today, sequencing a micro-library of genomic fragments with sufficient coverage can be accomplished in one or at most several days. Coupled with strategies to identify those PKS fragments that correspond to the sought-after gene cluster, the method is especially useful when embarking on a search for a new PKS gene cluster.

Sequencing a specified number of fragments from a genomic library yields a predictable probability of obtaining a fragment from each and every modular PKS gene cluster in the genome; assurance is thus provided that a probe is present for a sought-after PKS gene cluster. The statistical information generated from the DNA sequencing effort allows an estimate of the fraction of the genome that contains modular PKS genes and, with the size of a typical PKS gene cluster, the approximate number of PKS gene clusters in the genome. The PKS fragments obtained from the sequencing effort can be used as “perfect probes” in experiments aimed at isolating a sought-after or all modular PKS gene clusters in an organism. Use of the approach described here indicates that ˜3.2% of the Sorangzum cellulosum SMP44 genome or a total of ˜320 kb corresponds to PKS gene sequences. In addition to the two known PKS gene clusters in the genome, there may be four to six others. The approach may not be as practical for the smaller non-modular PKS gene clusters but is applicable to the analysis of NRPS gene clusters.

The methods of the present invention constitute a significant advance over prior art methods for identifying and cloning PKS and NRPS genes and gene clusters. In the prior art methods, such genes and gene clusters were typically identified in genomic libraries by probing with degenerate or other probes derived from known PKS or NRPS genes. Using the methods of the present invention, one obtains probes that are perfectly complimentary, and so are called perfect probes, to the PKS or NRPS gene or gene clusters of interest. Moreover, these perfect probes are obtained simply by sequencing a limited number, the “micro-library”, of randomly generated genomic clones. In one embodiment, the invention provides a method for generating perfect probes to PKS or NRPS gene or gene clusters in an organism by sequencing insert nucleic acid from a number of genomic clones, the number being equal to the size, in kilobases, of an average PKS or NRPS gene or gene cluster divided by the size, in kilobases, of the genome of the organism times 100. In more preferred embodiments, from two to five times this number of clones is sequenced, thus increasing the probability that all PKS or NRPS genes or gene clusters in an organism are represented in the sequenced microlibrary. For identification of PKS gene clusters, the average size will generally be in the range of 30 to 100 kb, the insert size of inserts in the genomic library is ideally in the range of 1 to 5 kb, although insert size can be larger or smaller, i.e., in the range of 0.25 to 10 kb. Typically one obtains at least about 100 to 500 nucleotides of sequence information from each insert sequenced, preferably 200 to 300 nucleotides of sequence.

The following examples are given for the purpose of illustrating the present invention and shall not be construed as being a limitation on the scope of the invention or claims.

### EXAMPLES

Sorangium cellulosum strain SMP44 produced epothilones A and B as determined by HPLC/MS. Genomic DNA was prepared as described (Jaoua et al., Plasmid 28 (1992) 157-165); the DNA was fragmented by nebulization, size selected for ˜1 to 2 kb fragments and cloned into the SmaI site of pUC18 (Bodenteich et al., in Adam et al. (eds.), Automated DNA Sequencing and Analysis Techniques. Academic Press, London, 1994, pp. 42-50; Roe, http://www.genome.ou.edu, 1999). Sequencing was performed using reverse and forward universal primers on an ABI 377 DNA sequencer, with confirmation on a Beckman CEQ2000 capillary sequencer, to give 495 readable sequences. A PERL script (Wall et al., Programming Perl. O'Reilly, Sebastopol, 1991), running on Unix, was used to automate the BLAST searches (Altschul et al., Nucleic Acids Res. 25 (1997) 3389-3402) of S. cellulosum sequences against the NCBI non-redundant database. The script feeds the sequences into the NCBI BLAST site (http://www.ncbi.nlm.nih.gov/blast/blast.cgi?Tform=0), and each submission returns a set of alignments to the PERL script in order of increasing P-value; it then scans the 20 best alignments for PKS and NRPS annotations. A P-value of at least e⁻²⁰ against known PKS or NRPS genes was required before domain assignment was pursued.

The invention having now been described by way of written description and examples, those of skill in the art will recognize that the invention can be practiced in a variety of embodiments and that the foregoing description and examples are for purposes of illustration and not limitation of the following claims. All patent applications and publications cited herein are hereby incorporated herein by reference.

1. A method for generating a perfect probe for any PKS or NRPS gene or gene cluster in an organism, the method comprising the steps of:
(a) generating a genomic library of vectors containing insert DNA from said organism;
(b) generating nucleotide sequence information from said vectors;
(c) comparing said nucleotide sequence information generated with sequence information from a known PKS or NRPS gene; and
(d) identifying vectors with insert DNA that contains nucleotide sequences from a PKS or NRPS gene,
wherein said insert DNA that contains nucleotide sequences from a PKS or NRPS gene is a perfect probe for said PKS or NRPS gene.

2. The method of claim 1, wherein a set of perfect probes comprising at least one perfect probe for each PKS or NRPS gene cluster in the genome of said organism.

3. The method of claim 1, wherein said perfect probe is used to identify by hybridization clones in a genomic library containing very large inserts of genomic DNA of the organism that contain the PKS or NRPS genes of interest.

4. The method of claim 3, wherein said genomic library is a BAC or cosmid library.

5. The method of claim 1, wherein sequence information is obtained from all clones in a micro-library.

6. The method of claim 5, wherein sequence information is obtained from a number of clones that is two times the number of clones in a micro-library.

7. The method of claim 6, wherein sequence information is obtained from a number of clones that is three times the number of clones in a micro-library.

8. The method of claim 7, wherein sequence information is obtained from a number of clones that is four times the number of clones in a micro-library.

9. The method of claim 8, wherein sequence information is obtained from a number of clones that is five times the number of clones in a micro-library.

10. The method of claim 9, wherein sequence information is obtained from clones containing inserts identical to at least a portion of each PKS or NRPS gene cluster in said organism.

11. The method of claim 10, wherein one or more oligonucleotides complementary to one or more inserts identical to at least a portion of each PKS or NRPS gene cluster are synthesized.

12. The method of claim 11, wherein a set of oligonucleotide is synthesized, said set comprising at least one probe complementary to each PKS or NRPS gene cluster.

13. The method of claim 11, wherein said oligonucleotide is used to identify DNA fragments, recombinant vectors, or host cells comprising all or a portion of the PKS or NRPS gene cluster.

14. The method of claim 11, wherein said oligonucleotide is used to amplify a DNA or RNA derived from the PKS or NRPS gene cluster.

15. The method of claim 13, wherein a recombinant vector comprising at least one gene of said gene cluster is identified, and the nucleotide sequence of said genes is determined.
